(19) United States
(12) Patent Application Publication
(10) Pub. No.: US 2026/0108552 A1
(43) Pub. Date: Apr. 23, 2026

(54) Uses of Dendrobium polysaccharides and vancomycin-sensitive bacteria, and compositions containing the same

(71) Applicant: Hong Kong Authentication Centre of Valuable Chinese Medicines, Hong Kong (CN)

(72) Inventors: Quanbin HAN, Hong Kong (CN); Chuying HUO, Hong Kong (CN); Lifeng LI, Hong Kong (CN)

(73) Assignee: Hong Kong Authentication Centre of Valuable Chinese Medicines, Hong Kong (CN)

(21) Appl. No.: 19/306,545

(22) Filed: Aug. 21, 2025

Related U.S. Application Data
(60) Provisional application No. 63/685,480, filed on Aug. 21, 2024.

(51) Int. Cl. A61K 31/715; A61K 35/00; A61K 35/745; A61K 36/8984; A61K 38/47; A61P 1/04

(52) U.S. Cl. CPC A61K 31/715; A61K 35/745; A61K 36/8984; A61K 38/47; A61P 1/04; C12Y 302/01078; A61K 2035/115

(57) ABSTRACT

Disclosed are beneficial uses of Dendrobium polysaccharides, and compositions comprising them and vancomycin-sensitive bacteria for intestinal disorders. The present disclosure relates to a method of treating an intestinal disorders, such as an inflammatory bowel disease, comprising administering Dendrobium polysaccharide to an individual in need thereof. The present disclosure also relates to a method of treating an intestinal disease comprising administering vancomycin-sensitive bacteria to an individual in need thereof, wherein said individual has taken or is about to take Dendrobium polysaccharide.

### CROSS-REFERENCE TO RELATED APPLICATION

This application claims the benefit of priority to U.S. Provisional Patent Application No. 63/685,480, filed on Aug. 21, 2024, the entire contents of which are hereby incorporated by reference.

### INCORPORATION BY REFERENCE OF SEQUENCE LISTING

The Sequence Listing in an XML format, named as 43664_SubstituteSequenceListing.xml of 8,339 bytes, created on Nov. 26, 2025, and submitted to the United States Patent and Trademark Office via Patent Center, is incorporated herein by reference.

### FIELD OF THE INVENTION

The present disclosure is related to uses of Dendrobium polysaccharides and vancomycin-sensitive bacteria, and compositions comprising combinations of Dendrobium polysaccharides, specifically, Dendrobii β-1,4-mannan (DOP), and probiotic lysates derived from vancomycin-sensitive bacteria that are responsible for the metabolism of DOP. In particular, the vancomycin-sensitive bacteria Bifidobacterium pseudolongum, is capable of degrading DOP into mannan oligosaccharides and contributes to the beneficial effects of DOP.

The compositions are useful in the treatment of inflammatory bowel disease and restoration of intestinal microbial balance.

### BACKGROUND OF THE INVENTION

Ulcerative colitis, a common inflammatory bowel disease affecting the large intestine, is characterized by symptoms such as abdominal pain, weight loss, rectal bleeding, and diarrhea (Le Berre et al., 2023).

Dendrobium species have been used as a functional food in China for centuries. Dendrobium polysaccharides (DP) is found in the stem of genus Dendrobium, a traditional Chinese medicine, and has been shown to have a variety of biological functions. The polysaccharides extracted from Dendrobium officinale (Dendrobium officinale polysaccharide, DOP) has attracted researchers' interest for its various bioactivities, such as anti-tumor, immunoregulatory, anti-oxidant, anti-diabetes, and neuroprotective effects.

Studies have shown the anti-colitis effects of DOP, including symptom relief, reduced inflammation, and enhanced intestinal barrier function. Despite the recent advancements in documenting its beneficial effects, the underlying mechanisms of these effects remain unclear.

### SUMMARY OF THE INVENTION

The present application will be further explained below in detail through embodiments. Through these descriptions, the features and advantages of the present application will become more clearly defined.

The term “exemplary” used here means “serving as an example, embodiment, or illustration.” Any embodiment described here as “exemplary” should not be interpreted as being superior or better than other embodiments.

Furthermore, the technical features in different embodiments of the present application described below can be combined with each other if they do not conflict.

The following embodiments are provided to illustrate the purpose and should not limit the present disclosure in any way. One of skill in the art should easily recognize that various non-critical parameters can be changed or modified to obtain essentially the same results.

The backbone of Dendrobium officinale polysaccharide (DOP) was reported to be glucomannan. As a result of the applicant's diligent research, the backbone structure of DOP is determined to be β-D-1,4-mannan, which differs from the previous studies. It was also confirmed that the DOP prepared from multiple batches of herbal materials presented highly consistent molecular size and oligosaccharide profiles.

In the present disclosure, the applicants of the present disclosure investigates the protective effect of DOP (having the backbone structure of β-D-1,4-mannan) against colitis, and the contribution of gut microbiota to DOP's protective effects on colitis.

The role of gut microbiota in DOP's bioactivities has not been fully studied. DOP was found to be non-absorbed and undegraded in the stomach and small intestine until it reached the large intestine where it was fermented by gut microbiota.

The applicant used different antibiotics as diagnostic reagents to classify the mediating bacteria and found that antibiotic cocktail-induced depletion of bacteria eliminated DOP's degradation. Vancomycin (rather than neomycin)-sensitive bacteria mediated the degradation of DOP and its anti-colitis effect.

The increased expression of mannan endo-1,4-mannosidase (EC 3.2.1.78) together with stronger catalyzing ability in DOP degradation were observed in DOP-treated microbiota. This enzyme can break down DOP but is depleted by vancomycin.

Further, the applicant identified vancomycin-sensitive Bifidobacterium pseudolongum, which cleaves DOP to generate mannan oligosaccharides, as a key contributor to DOP fermentation and the anti-colitis effects of DOP.

In summary, the applicant found that the breakdown of DOP by mannan endo-1,4-β-mannosidase in vancomycin-sensitive Bifidobacterium pseudolongum plays a crucial role in its anti-colitis effects, and as a result, the applicant found methods to increase the level of utilization of DOP by mammal.

A main embodiment of the present disclosure is a combination of (i) Dendrobium polysaccharides; and (ii) cells of bacteria, or probiotic lysate, or a composition comprising a combination of
(i) Dendrobium polysaccharides; and
(ii) cells of bacteria, or probiotic lysate,

wherein the probiotic lysate is derived from at least one species of vancomycin-sensitive bacteria, or the bacteria is vancomycin-sensitive bacteria.

In one embodiment, said Dendrobium polysaccharide (DP) is an extract of plant Dendrobium. An example of said plant Dendrobium includes but not limited to: Dendrobium officinale, Dendrobium nobile Lindl, Dendrobium wardianum, Dendrobium Second Love ‘Tokimeki’.

In one embodiment, the extract of Dendrobium is extracted from a stem of Dendrobium, said stem being either a dried substance of a stem or a stem of a fresh plant.

In one embodiment, said Dendrobium polysaccharide (DP) is Dendrobium officinale polysaccharide (DOP).

In one embodiment, the vancomycin-sensitive bacteria include following genera: Schaedlerella, Acetatifactor, Enterocloster, Bacteroides, Alistipes, Vermiculatibacterium, Lactobacillus, Roseburia, Eubacterium, Lachnoclostridium, Phocaeicola, Prevotella, Anaerotruncus, Muribaculum, Duncaniella, Faecalibaculum, Adlercreutzia, Odoribacter, Bifidobacterium, Barnesiella, Dubosiella, Butyricicoccus, Candidatus, Amulumruptor, Flavonifractor, Ileibacterium, Allobaculum, Otoolea, Hungatella, Neglectibacter, Oscillibacter, Acutalibacter, and Palleniella.

In one embodiment, said vancomycin-sensitive bacteria is Bifidobacterium pseudolongum. In one embodiment, the Bifidobacterium pseudolongum includes at least one selected from DSM20092 and DSM20099.

In one embodiment, the extract contains bacterial proteins, and said bacterial proteins is total proteins of bacteria. In one embodiment, said bacterial proteins contain carbohydrate-active enzymes (CAZymes). The carbohydrate-active enzymes includes but not limited to: a glycoside hydrolase. In one embodiment, the glycoside hydrolase is EC 3.2.1.78 enzyme. Another embodiment of the present disclosure is a use of vancomycin-sensitive bacteria, comprising
making therapeutically effective doses of vancomycin-sensitive bacteria available in the intestines of subjects in need thereof, for increasing intestinal absorption of Dendrobium polysaccharide (DP) in subjects in need thereof.

In one embodiment, making therapeutically effective doses of vancomycin-sensitive bacteria available in the intestines of subjects in need thereof comprises one or more of the following:
discontinuing vancomycin;
administering DOP;
supplementing with probiotics; and
restoring the subject's intestinal flora balance.

In one embodiment, the subjects in need thereof have an inflammatory bowel disease.

Another embodiment of the present disclosure is A composition for increasing gut health, comprising
(i) Dendrobium polysaccharide (DP); and
(ii) live, inactivated, dead, or lysed bacteria of vancomycin-sensitive bacteria, etc.

In one embodiment, the increasing gut health comprises one or more of the following:
restoring dysbiosis in a subject's gut microbial ecosystem, increasing a subject's intestinal short-chain fatty acid (SCFA) production, increasing mannan oligosaccharide production, alleviating colitis-like symptoms, restoring colon length, suppressing intestinal inflammation, enhancing intestinal barrier function, and decreasing colonic injury.

Another embodiment of the present disclosure is a method for treating an inflammatory bowel disease in a subject, comprising:
i) administering a therapeutically effective dose of DP;
ii) prior to step i,
making therapeutically effective doses of vancomycin-sensitive bacteria available in the intestines of the subject, wherein the vancomycin-sensitive bacteria express glycoside hydrolase EC 3.2.1.78; or
administering a therapeutically effective dose of bacterial proteins of the vancomycin-sensitive bacteria, and/or glycoside hydrolase EC 3.2.1.78 to the subject.

In one embodiment, making therapeutically effective doses of vancomycin-sensitive bacteria available in the intestines of the subject comprises:
administering a therapeutically effective dose of vancomycin-sensitive bacteria to the subject.

In one embodiment, the inflammatory bowel disease is selected from: indeterminate colitis, ulcerative colitis and Crohn's disease.

In one embodiment, alleviated DSS-induced colitis can be evidenced by the alleviated colitis-like symptoms, restored colon length, suppressed intestinal inflammation, enhanced intestinal barrier function, and reduced colonic injury.

In one embodiment, the inflammatory bowel disease related indicators or colitis-related indicators may include body weight loss, diarrhea, rectal bleeding, increased intestinal inflammation, disrupted intestinal barrier function, and colonic histopathological changes.

Another embodiment of the present disclosure is a method for preventing or treating inflammatory bowel disease comprising:
i) a testing step,
the testing step testing for the presence of a therapeutically effective dose of vancomycin-sensitive bacteria in the intestinal tract of a subject in need thereof;
ii) a supplementing or transplanting bacteria or restoring intestinal flora balance step,
wherein when an effective amount of vancomycin-sensitive bacteria is not detected in the testing step of 1), supplement the subject with probiotics or restoring the intestinal flora balance in the subject, and carry out the testing step again;
iii) a polysaccharide supplementation step,
wherein when an effective amount of vancomycin-sensitive bacteria is detected in the testing step of i) or ii), administer a therapeutically effective amount of DP to the subject.

In one embodiment, the vancomycin-sensitive bacteria includes one or more of the following genera: Schaedlerella, Acetatifactor, Enterocloster, Bacteroides, Alistipes, Vermiculatibacterium, Lactobacillus, Roseburia, Eubacterium, Lachnoclostridium, Phocaeicola, Prevotella, Anaerotruncus, Muribaculum, Duncaniella, Faecalibaculum, Adlercreutzia, Odoribacter, Bifidobacterium, Barnesiella, Dubosiella, Butyricicoccus, Candidatus, Amulumruptor, Flavonifractor, Ileibacterium, Allobaculum, Otoolea, Hungatella, Neglectibacter, Oscillibacter, Acutalibacter, and Palleniella.

In one embodiment, said DP is Dendrobium officinale polysaccharide (DOP).

In one embodiment, the backbone structure of DOP is β-D-1,4-mannan.

Another embodiment of the present disclosure is a method for treating or preventing an inflammatory bowel disease in a subject, comprising:
i) administering a therapeutically effective dose of DOP,
wherein the backbone structure of DOP is β-D-1,4-mannan.

In one embodiment, said method further comprises:
ii) administering a therapeutically effective dose of vancomycin-sensitive bacteria,
wherein the vancomycin-sensitive bacteria includes one or more of the following genera: Schaedlerella, Acetatifactor, Enterocloster, Bacteroides, Alistipes, Vermiculatibacterium, Lactobacillus, Roseburia, Eubacterium, Lachnoclostridium, Phocaeicola, Prevotella, Anaerotruncus, Muribaculum, Duncaniella, Faecalibaculum, Adlercreutzia, Odoribacter, Bifidobacterium, Barnesiella, Dubosiella, Butyricicoccus, Candidatus, Amulumruptor, Flavonifractor, Ileibacterium, Allobaculum, Otoolea, Hungatella, Neglectibacter, Oscillibacter, Acutalibacter, and Palleniella.

Another embodiment of the present disclosure is the use of the composition according to the main embodiment in use for restoring intestinal flora dysbiosis in a subject.

Another embodiment of the present disclosure is the use of the composition according to the main embodiment in the use for elevating intestinal SCFA production in a subject.

In one embodiment, the SCFA includes but not limited to: acetate, butyrate and propionate. Another embodiment of the present disclosure is the use of the composition according to the main embodiment in the treatment or prevention of an inflammatory bowel disease in a subject.

Another embodiment of the present disclosure is the use of Bifidobacterium pseudolongum in the preparation of adjunctive foodstuffs and/or medicines. The adjunctive foodstuffs and/or medicines may be administered to a subject while the subject is receiving or about to receive Dendrobium polysaccharide for the treatment of colitis.

Another embodiment of the present disclosure is the use of EC 3.2.1.78 in the preparation of adjunctive foodstuffs and/or medicines. The adjunctive foodstuffs and/or medicines may be administered to a subject while the subject is taking, or about to take Dendrobium polysaccharide for the treatment of colitis.

Another embodiment of the present disclosure is a method for use in restoring a) intestinal dysbiosis or aa) gut microbiota imbalance in a subject, comprising:
i) administering a therapeutically effective dose of Dendrobium polysaccharides; and
ii) administering a therapeutically effective dose of vancomycin-sensitive bacteria, bacterial proteins of vancomycin-sensitive bacteria, or EC 3.2.1.78.

Another embodiment of the present disclosure is a method for treating or preventing an inflammatory bowel disease in a subject, comprising:
i) administering a therapeutically effective dose of Dendrobium polysaccharide; and
ii) administering a therapeutically effective dose of a vancomycin-sensitive bacteria, a bacterial protein of a vancomycin-sensitive bacteria, or EC 3.2.1.78.

In one embodiment, the DOP is prepared by the following method:
a) the dried stem of Dendrobium was boiled twice with distilled water to obtain the extracts;
b) the extracts were combined and centrifuged, and the supernatant was obtained and then concentrated by a rotary evaporator;
c) the concentrated extracts of b) were mixed with ethanol (4:6, v/v) for overnight precipitation;
d) the mixture of c) was centrifuged, and the sediment was collected and washed with ethanol;
e) the sediment of d) was redissolved for de-starching, add heat-stable α-amylase, followed by incubation at 80° C., and the enzymatically hydrolyzed sediment was obtained,
f) utilize the Sevage method to remove proteins from the enzymatically hydrolyzed sediment of e), employing a DOP/chloroform/butanol ratio of 25/5/1 and repeating the extraction process;
g) evaporate the organic solvents using rotary evaporation;
h) dialysis to remove substances lower than 10.0 kDa molecular weight;
i) centrifuge the product of h), and
j) the supernatant of f) was collected and lyophilized.

In one embodiment, the DOP is prepared by the following method:
a) the dried stem of Dendrobium officinale was boiled twice with distilled water at 100° C. for 2 h;
b) the extracts were combined and centrifuged at 4,000 rpm for 10 min, and the supernatant was obtained and then concentrated by a rotary evaporator;
c) the concentrated extracts were mixed with ethanol (4:6, v/v) for overnight precipitation;
d) the mixture was centrifuged, and the sediment was collected and washed with 70% ethanol;
e) the sediment was redissolved in distilled water for de-starching, and add heat-stable α-amylase, followed by incubation at 80° C.;
f) utilize the Sevage method to remove proteins from the enzymatically hydrolyzed sediment (DOP), employing a DOP/chloroform/butanol ratio of 25/5/1 and repeating the extraction process;
g) evaporate the organic solvents using rotary evaporation;
h) dialysis to remove substances lower than 10.0 kDa molecular weight;
i) the sample was centrifuged again at 4,000 rpm for 10 min; and
j) the supernatant was collected and lyophilized.

In one embodiment, the Dendrobium polysaccharide in said composition may be in the form of: dry powders, lyophilized powders, micronized powders or solution dissolved in water. The probiotic lysate in said composition may be in the form of: dry powders, lyophilized powders, micronized powders or solution dissolved in water.

In one embodiment, the composition can be prepared by mixing dry powder, lyophilized powder, micronized powder of the Dendrobium polysaccharide with dry powders, lyophilized powders, micronized powders or solution dissolved in water of the probiotic lysate.

In one embodiment, the subject is a mammal. Preferably, the subject is primates, more preferably, a human.

In one embodiment, said inflammatory bowel disease includes but not limited to: indeterminate colitis, ulcerative colitis and Crohn's disease. Preferably, inflammatory bowel disease is ulcerative colitis.

Another embodiment of the present disclosure is a method, for use in the treatment or prevention of colitis-associated cancer in a subject comprising administering a Dendrobium polysaccharide and either administering a vancomycin-sensitive bacteria, a bacterial protein of a vancomycin-sensitive bacteria, or an EC 3.2.1.78 enzyme.

In one embodiment, said colitis-associated cancer includes but not limited to: colitis-associated colorectal cancer (CAC).

Another embodiment of the present disclosure is the use of vancomycin for screening gut microorganisms that aid in the anti-colitis function of Dendrobium polysaccharides.

In one embodiment, the SCFA comprises: acetate, butyrate and propionate.

Another embodiment of the present disclosure is a method for increasing mannan oligosaccharide production in the intestines of subjects administered Dendrobium stercoralis polysaccharides, comprising:

administering a therapeutically effective dose of vancomycin-sensitive bacteria, bacterial proteins of vancomycin-sensitive bacteria, and/or EC 3.2.1.78.

In one embodiment, the composition is administered via a gastrointestinal route.

In one embodiment, it is recommended that vancomycin not be administered to the subject for a period of 2 weeks to 1 month before and after the administration of said composition.

### Advantages of the Invention

The composition of the present disclosure solves the problem of non-absorption of Dendrobium polysaccharides. The compositions of the present disclosure could greatly improve the degradation rate of Dendrobium polysaccharides in the intestinal tract.

The compositions of the present disclosure enhance the reparative function of Dendrobium polysaccharides on the intestinal tract by increasing the level of degradation (or, breakdown) products of Dendrobium polysaccharides in the intestinal tract.

The main ingredients of the compositions of the present disclosure are derived from food-like plants that have been used for many years, and have a good safety profile.

The microbial component contained in the composition of the disclosure is also derived from natural microorganisms that can survive in the intestinal tract, have advantages in terms of safety. For example, DSM20092 and DSM20099 are able to survive in the intestinal tract of humans and mice.

The compositions of the present disclosure may be consumed as food or as ingredients added to food.

In addition to the alleviating and therapeutic effect on inflammatory bowel disease in the subject, said composition has a variety of beneficial effects on the organism, for example, it also has anti-tumor, immunoregulatory, anti-oxidant, anti-diabetes, and neuroprotective effects.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 shows DOP ameliorated DSS-induced colitis in mice. (FIG. 1A) Schedule of experiment. (FIG. 1B) Bodyweight changes during the DSS challenge. (FIG. 1C) Disease activity index (DAI) during DSS challenge. (FIG. 1D) Representative images of the colon. (FIG. 1E) Statistical analysis of colon length. (FIG. 1F) Expression of LCN2 in the feces. (FIG. 1G) Expression of IL-6 in the colon. (FIG. 1H) Expression of TNF-α in the colon. (FIG. 1I) Representative images of H&E stained colon sections. (FIG. 1J) Histopathological score of the colon sections. *P<0.05. **P<0.01. ****P<0.0001. ^{#}P<0.05, ^{##}P<0.01, ^{####}P<0.0001, versus the DSS group.

FIG. 2 shows DOP treatment enhanced the intestinal tight junction in colitis mice. (FIG. 2A) Representative images of colon sections. (FIG. 2B) Statistical analysis of the fluorescent intensity of ZO-1. (FIG. 2C-FIG. 2E) Relative mRNA levels of zo-1 (FIG. 2C), occludin (FIG. 2D), and muc2 (FIG. 2E) (normalized to β-actin) in the colon tissue. *P<0.05, **P<0.01.

FIG. 3 shows DOP changed the composition of gut microbiota in colitis mice. (FIG. 3A) PCA analysis. (FIG. 3B) Relative abundance at the genus level. (FIG. 3C) Relative abundance comparison of the genera Bacteroides, Parabacteroides, Intestinimonas, Flavonifractor, Eubacterium, Escherichia, and Alloprevotella. (FIG. 3D) Differentially abundant species comparing the DSS group and DSS+DOP group based on the Wilcoxon rank-sum test. Left panel: Log 2 value of the mean relative abundance ratio of the species. Middle panel: P value and FDR of the species obtained using the Wilcox test. Right panel: Correlation analysis of these species with the colitis-related indexes. The marked species indicate that this species was abundant in the DSS+DOP group and related to the anti-colitis effect of DOP. *P<0.05. **P<0.01, P<0.001, ****P<0.0001.

FIG. 4 shows DOP increased the production of SCFAs in colitis mice. (FIG. 4A-FIG. 4D) Relative peak area of total SCFAs (FIG. 4A), acetate (FIG. 4B), butyrate (FIG. 4C), and propionate (FIG. 4D) in the cecum of colitis mice. (FIG. 4E) Correlation analysis between the content of SCFAs and colitis-related indexes. *P<0.05, **P<0.01. ***P<0.001, **** P<0.0001.

FIG. 5 shows Vancomycin-sensitive bacteria are responsible for the fermentation of DOP. (FIG. 5A-FIG. 5E) Mice were treated with normal drinking water, ABX, vancomycin, and neomycin in drinking water, respectively. DOP was orally administrated to mice simultaneously. (FIG. 5A) Experimental schedule. (FIG. 5B-FIG. 5E) Relative peak area of total SCFAs (FIG. 5B), acetate (FIG. 5C), butyrate (FIG. 5D), and propionate (FIG. 5E) in the cecum were measured. (FIG. 5F-FIG. 5K) Mice were treated with normal drinking water, vancomycin, or neomycin for one week. A cecum loop experiment was carried out by injecting the FITC-DOP into the cecum and incubating it for one hour. (FIG. 5F) Experimental design. (FIG. 5G) HPGPC-FLD chromatogram of F-DOP in these groups. The dotted line shows the chromatographic retention time of F-DOP. (FIG. 5H-FIG. 5K) Relative content of total SCFAs (FIG. 5H), acetate (FIG. 5I), butyrate (FIG. 5J), and propionate (FIG. 5K) were measured. *P<0.05, **P<0.01. ***P<0.001. ****P<0.0001.

FIG. 6 shows DOP's anti-colitis effect was abolished by vancomycin. (FIG. 6A) Schedule of experiment. (FIG. 6B) Body weight change during DSS challenge. (FIG. 6C) DAI in these groups. (FIG. 6D) Statistical analysis of colon length. (FIG. 6E) Expression of IL-6 in colon. (FIG. 6F) Expression of TNF-α in colon. (FIG. 6G) Representative images of H&E staining of the colon sections. (FIG. 6H) Histological score of the colon. (FIG. 6I-FIG. 6L) Relative peak area of total SCFAs (FIG. 6I), acetate (FIG. 6J), butyrate (FIG. 6K), and propionate (FIG. 6L) in the cecum of mice. *P<0.05. **P<0.01. ***P<0.001, ****P<0.0001.

FIG. 7 shows Vancomycin-induced depletion of EC 3.2.1.78 was related to the fermentation and bioactivity of DOP. (FIG. 7A-C) Bacterial protein was extracted from the cecal content of the Control group, DSS group, and DSS+DOP group. The bacterial protein was then incubated with F-DOP for 6 h. (FIG. 7A) Experimental schedule. (FIG. 7B) HPGPC-FLD chromatogram of F-DOP after incubation with the bacterial protein from the Control group, DSS group, and DSS+DOP group. The dotted line shows the chromatographic retention time of F-DOP incubated with the bacterial protein from the Control group. (FIG. 7C) LC-MS chromatograms of oligosaccharides produced by incubation of F-DOP with the bacterial protein from these three groups. Indicated peaks are ABEE-labeled mannan oligosaccharides. Man, mannose. (FIG. 7D) Differentially expressed CAZymes in control mice (Control) and vancomycin-treated mice (Vancomycin). (FIG. 7E) The LC-MS chromatogram of oligosaccharides produced by incubation of DOP with the EC 3.2.1.78. (FIG. 7F) Relative expression of EC 3.2.1.78 between the control mice and vancomycin-treated mice. (FIG. 7G) Relative expression of EC 3.2.1.78 between the DSS group and DSS+DOP group. (FIG. 7H) Bacterial protein was extracted from control mice and vancomycin-treated mice. Left panel: Coomassie blue staining. Right panel: zymography of bacterial protein in degrading DOP. Endo-1,4-β-mannosidase (EC 3.2.1.78) was used as a positive control. ****P<0.0001.

FIG. 8 shows Vancomycin-sensitive Bifidobacterium pseudolongum is involved in the degradation of DOP. (FIG. 8A-FIG. 8G) Gut microbial profiles of the control mice (Control, C) and vancomycin-treated mice (Vancomycin, V). (FIG. 8A) Number of non-redundant genes. (FIG. 8B) Venn diagram. (FIG. 8C) PCA analysis. (FIG. 8D-FIG. 8E) An OTU hierarchical clustering tree representing the distribution of microbiota at the phylum level (FIG. 8D) and the genus level (FIG. 8E) based on Bray-Curtis dissimilarities. (FIG. 8F)

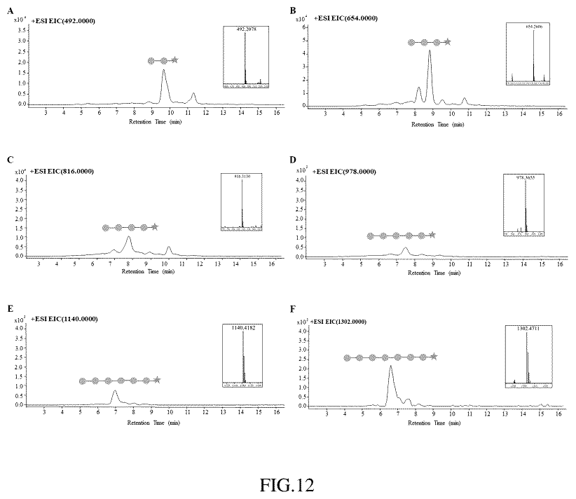

Metastat cluster analysis heatmap showing the significantly changed bacteria at the species level. Marked species are those related to the development of ulcerative colitis. (FIG. 8G) Relative abundance of B. pseudolongum in control mice and vancomycin-treated mice. (FIG. 8H) Relative abundance of B. pseudolongum in the DSS group and DSS+DOP group. (FIG. 8I) HPGPC-FLD chromatogram of F-DOP after incubation with the bacterial protein from B. pseudolongum and the cecum of control mice for 24 h. The dotted line represents the chromatographic retention time of intact F-DOP. (FIG. 8J) The LC-MS chromatogram of oligosaccharides. Upper panel: DOP incubated with the bacterial protein from B. pseudolongum. Middle panel: DOP. Lower panel: the bacterial protein from B. pseudolongum. ****P<0.0001.

FIG. 9 shows Gut microbiota profile regulated by DOP in the colitis mice. The alpha diversity of gut microbiota was shown by the Sobs (FIG. 9A), ACE (FIG. 9B), Chao (FIG. 9C), Shannon diversity index (FIG. 9D), and Simpson diversity index (FIG. 9E). (FIG. 9F) Relative abundance at the phylum level.

FIG. 10 shows LefSe analysis of the gut microbiota between the normal mice and vancomycin-treated mice (FIG. 10A), and the significantly different abundant genus between the Control group and the Vancomycin group (FIG. 10B).

The significantly different abundant genera between the Control group and the Vancomycin group (P<0.05): Schaedlerella, Acetatifactor, Enterocloster, Bacteroides, Alistipes, Vermiculatibacterium, Lactobacillus, Roseburia, Eubacterium, Lachnoclostridium, Phocaeicola, Prevotella, Anaerotruncus, Muribaculum, Duncaniella, Faecalibaculum, Adlercreutzia, Odoribacter, Bifidobacterium, Barnesiella, Dubosiella, Butyricicoccus, Candidatus, Amulumruptor, Flavonifractor, Ileibacterium, Allobaculum, Otoolea, Hungatella, Neglectibacter, Oscillibacter, Acutalibacter and Palleniella.

FIG. 11 shows B. pseudolongum mediated degradation of F-DOP. (FIG. 11A) Experimental design. B. pseudolongum and L. bacterium were cultured under the anaerobic condition, with F-DOP supplemented in the medium. F-DOP in the blank culture medium was used as a control. The incubation was conducted in a shaking incubator. (FIG. 11B-FIG. 11D) HPGPC-FLD histogram of F-DOP after incubation with different strains of B. pseudolongum: DSM 20092 (FIG. 11B), and DSM 20099 (FIG. 11C). The dotted line represented the retention time of the intact F-DOP. (FIG. 11D-FIG. 11F) HPGPC-FLD histogram of F-DOP after incubation with different strains of L. bacterium: DSM 111138 (FIG. 11D), DSM 24404 (FIG. 11E), DSM 24430 (FIG. 11F). The dotted line represented the retention time of the intact F-DOP.

FIG. 12 shows EICs (ESI⁺) of mannan oligosaccharides extracted from the incubation of DOP with the bacterial protein from B. pseudolongum. (FIG. 12A) Mannan disaccharide. (FIG. 12B) Mannan trisaccharide. (FIG. 12C) Mannan tetrasaccharide. (FIG. 12D) Mannan pentasaccharide. (FIG. 12E) Mannan hexasaccharide. (FIG. 12F) Mannan heptasaccharide.

### DETAILED DESCRIPTION OF THE INVENTION

### Definitions

Dendrobium polysaccharides (DP) is the polysaccharide component extracted from the dried stem of Dendrobium.

In this disclosure, Dendrobium officinale polysaccharides is used in an equivalent sense to DOP, the polysaccharide component extracted from the stem of Dendrobium officinale Kimura et Migo. DOP refers to Dendrobii β-1,4-mannan, the β-1,4-manna from Dendrobium officinale. Experiments have shown that the DOP prepared from multiple batches of herbal materials presented highly consistent molecular size and oligosaccharide profiles.

It is to be noted that while there are significant differences in molecular weight, monosaccharide composition, and linkage types of DOP reported in other previous reports (He et al., 2016; Hua et al., 2004; Kuang et al., 2020; Luo et al., 2016; Wei et al., 2016; Xing et al., 2014; Yang et al., 2020), it is suggested that the DOP samples studied in these published works, should be close to the same substance, that having the backbone structure of β-D-1,4-mannan.

Vancomycin-sensitive bacteria are microbiota whose growth is suppressed by vancomycin.

In this disclosure, the vancomycin-sensitive bacteria includes one or more of the following genera: Schaedlerella, Acetatifactor, Enterocloster, Bacteroides, Alistipes, Vermiculatibacterium, Lactobacillus, Roseburia, Eubacterium, Lachnoclostridium, Phocaeicola, Prevotella, Anaerotruncus, Muribaculum, Duncaniella, Faecalibaculum, Adlercreutzia, Odoribacter, Bifidobacterium, Barnesiella, Dubosiella, Butyricicoccus, Candidatus, Amulumruptor, Flavonifractor, Ileibacterium, Allobaculum, Otoolea, Hungatella, Neglectibacter, Oscillibacter, Acutalibacter, and Palleniella.

Inflammatory bowel disease (IBD) includes ulcerative colitis and Crohn's disease.

Ulcerative colitis (UC) is a common inflammatory bowel disease that affects the large intestine. It is characterized by abdominal pain, diarrhea, rectal bleeding, weight loss, and fatigue. Probiotic lysate is total components, including total protein, extracted from vancomycin-sensitive bacteria.

Anti-colitis function refers to the decreased expression of pro-inflammatory cytokines, such as IL-6 and TNF-α.

Colitis-associated cancer is a specific type of colorectal cancer that arises in the context of chronic inflammatory bowel disease like ulcerative colitis. The chronic inflammation in ulcerative colitis can lead to DNA damage and mutations, increasing the risk of colitis-associated cancer.

Colitis-associated colorectal cancer (CAC). Patients with IBD, including ulcerative colitis and Crohn's disease, are known to have an increased risk of developing CAC.

Restoring intestinal dysbiosis means rebalancing the intestinal microbial community to a healthy condition, including promoting the growth of probiotics and suppressing the relative abundance of harmful bacteria.

Lipocalin-2 (LCN2) is a biomarker for intestinal inflammation.

The supplementing or transplanting bacteria or restoring intestinal flora balance, can be done, for example, by giving the subject probiotics, performing Fecal Microbiota Transplantation, or any other known safe ways.

EC 3.2.1.78, commonly referred to as mannan endo-1,4-β-mannosidase or β-mannanase, is an enzyme that capable of randomly hydrolyzing the β-1,4-mannosidic bonds within the mannans.

Bifidobacterium pseudolongum is a gram-positive and nonmotile bacterium belonging to the Actinobacteria phylum, and it is commonly found in the gut microbiota of mammals.

The term “therapeutically effective dose” refers to the amount of the inventive compound that induces a biological or medical response in an individual, said response being, for example, a reduction in enzyme or protein activity, improvement of symptoms, remission of symptoms, delay or retardation of disease progression, or prevention of disease.

Bifidobacterium pseudolongum DSM20092 and DSM20099 were obtained from DSMZ (German Collection of Microorganisms and Cell Cultures GmbH). DSM20092 and DSM20099 are able to survive in the intestinal tract of humans and mice.

### EXAMPLES

The examples below illustrate the invention in greater detail.

Firstly, the applicant investigated the anti-colitis effects of DOP in the DSS-induced colitis model. The applicant further used antibiotic treatment to verify the role of gut microbiota in DOP's activity. The applicant compared the enzyme profiles of control mice and antibiotic-treated mice to determine the possible mediator for DOP degradation. Finally, the applicant identified one bacterium potentially involved in DOP fermentation and investigated its interaction with DOP.

### Example 1 DOP Protects from DSS-Induced Colitis in Mice

In this Example, the effect of DOP was evaluated in the DSS-induced colitis mice model.

Pro-inflammatory cytokines represent the inflammatory context of colitis. The expression of cytokines, including IL-6 and TNF-«, were measured in colon tissues. Lipocalin-2 (LCN2) is a biomarker for intestinal inflammation. The protein expression of LCN2 was elevated in the feces of colitis mice.

The intestinal epithelium, which acts as a physical barrier between antigens and mucosa, is associated with the severity of ulcerative colitis. Tight junction proteins have a pivotal role in the maintenance of intestinal integrity. Alternation of tight junctions may lead to a disrupted intestinal barrier, increased intestinal permeability, and eventually develop into a “leaky” gut. This allows the intestinal bacteria passing through the epithelium to trigger an inflammatory response.

Zonula occludens-1 (ZO-1), also known as tight junction protein-1. Mucin2 (muc2) mucin is the major component of the mucus layer which lies between the epithelium and lumen. ZO-1 and muc2. Changes in ZO-1 and muc2 levels were elevated in the DSS-induced colitis mice model.

### 1.1 Materials and Methods

The same materials and methods are used in all of the later Examples, if not otherwise indicated.

### 1.1.1 Antibodies and Reagents

Dried stem of authentic Dendrobium officinale was obtained from certified production areas in mainland China as reported (Wong et al., 2023). All ELISA kits were obtained from Biolegend (San Diego, CA, USA). Antibiotics were purchased from Meilun Biotechnology (Dalian, China). Dextran sulfate sodium (DSS, DB001) was purchased from TdB Labs (Ultuna, Sweden). The hematoxylin and Eosin Staining Kit was purchased from Beyotime (Jiangsu, China). Tissue-Tek O.C.T. compound was obtained from Sakura Finetek (Torrance, CA, USA).

Antibody against ZO-1 was purchased from Invitrogen (Thermo Fisher Scientific, Cleveland, OH, USA) and the goat anti-rabbit IgG antibody conjugated with Alexa Fluor 647 was purchased from Abcam (Cambridge, UK). 4′,6-Diamidine-2′-phenylindole dihydrochloride (DAPI) was purchased from Roche (Basel, Swiss). Fluorescein isothiocyanate isomer I (FITC), methyl sulphoxide (DMSO), 3-nitrophenylhydrazine hydrochloride (3-NPH-HCl), and 1-ethyl-3-(3-dimethylaminopropyl) carbodiimide hydrochloride (EDC.HCl) were purchased from Sigma (Saint Louis, MO, USA).

Heptanoic acid (HEPA), Hexanoic acid (HEXA), 2-Methylvaleric acid (2-MVA), 3-Methylpentanoic acid (3-MPA), 4-Methylvaleric acid (4-MVA), 2-Ethylbutyric acid (2-EBA), Valeric acid (VA), 2-Methylbutyric acid (2-MBA), Isobutyric acid (IBA), Butyric acid (BA), Propionic acid (PA), Acetic acid (AA) were purchased from Aladdin (Chicago, USA) with purity over 99%.

### 1.1.2 Preparation of DOP

The dried stem of Dendrobium officinale was boiled twice with distilled water at 100° C. for 2 h. The extracts were combined and centrifuged at 4,000 rpm for 10 min. The supernatant was obtained and then concentrated by a rotary evaporator. After that, the concentrated extracts were mixed with ethanol (4:6, v/v) for overnight precipitation. The mixture was centrifuged at 4,000 rpm for 10 min, and the sediment was collected and washed with 70% ethanol. Then, the sediment was redissolved in distilled water for de-starching. Dissolve 1 g of sediment in 500 mL of deionized water and add 1 mL of heat-stable α-amylase (Megazyme Ltd.), followed by incubation at 80° C. for 4 hours. Utilize the Sevage method to remove proteins from the enzymatically hydrolyzed sediment (DOP), employing a DOP/chloroform/butanol ratio of 25/5/1 and repeating the extraction process 6 times. Subsequently, evaporate the organic solvents using rotary evaporation. Followed by dialysis to remove substances lower than 10.0 kDa molecular weight. The sample was centrifuged again at 4,000 rpm for 10 min. The supernatant was collected and lyophilized to produce DOP.

### 1.1.3 Animal Experiments

Six-to-eight-week-old male C57BL/C mice were obtained from the Chinese University of Hong Kong and housed in a controlled environment with a 12-h/12-h dark-light cycle and free access to food and water. They were acclimated for one week before experiments began. All experiments with animals were carried out following the Animals Ordinance, Department of Health, Hong Kong Special Administration Region, China ((22-81) in DH/HT&A/8/2/6 Pt.5), for the care and use of experimental animals in compliance with ARRIVE guidelines.

In Example 1, to verify the effect of DOP on DSS-induced colitis, mice were divided into 3 groups: Control group, DSS group, and DSS+DOP group.

Mice in DSS+DOP group were treated with DOP (300 mg/kg) by oral gavage once a day for 2 weeks. All mice except those in the Control group were given 3% DSS in the drinking water for 1 week from Day 7 to Day 14.

Disease activity index was calculated by the score of body weight loss (none: 0; 1%-5%: 1; 5%-10%: 2; 10%-15%: 3; more than 15%: 4), stool consistency (normal: 0; loose stools: 2; watery diarrhea: 4), and rectal bleeding (no bleeding: 0; slight bleeding: 2; gross bleeding: 4). Feces samples were collected before mice were sacrificed. After mice were sacrificed, colon length was measured. Cecal content and colon were collected for further analysis.

### 1.1.4 H&E Staining

Colon tissue was embedded in the OCT compound and stored at −80° C. The frozen tissues were cut into 10 μm sections by a cryostat (Cryostar NX70, Thermo Fisher Scientific). Colon sections were air-dried and washed with water. Hematoxylin was applied to tissue sections for 10 min. After washing with running water, slides were stained with eosin, then washed to remove excess stain. Images were captured by a microscope (Leica DMI3000 B). For histological scoring see Table 1.

TABLE 1
Disease activity index calculation

| Score | Body weight loss | Stool consistency | Rectal bleeding |
|---|---|---|---|
| 0 | None | Normal | No bleeding |
| 1 | 1%-5% | | |
| 2 | 5%-10% | Loose stools | Slight bleeding |
| 3 | 10%-15% | | |
| 4 | More than 15% | Watery diarrhea | Gross bleeding |

### 1.1.5 Immunofluorescent Staining

Colon sections were air-dried and washed with water, then incubated with 2% goat serum for 1 h at room temperature, followed by anti-ZO-1 antibody (1:500) incubation overnight at 4° C. Then, sections were washed and incubated with secondary goat anti-rabbit IgG antibody conjugated with Alexa Fluor 647 (1:500) and DAPI (1 μg/mL) for 1 h at room temperature in the dark. After staining, the slides were sealed with an anti-fluorescence quenching reagent before being observed by a confocal microscope (Leica TCS SP8).

### 1.1.6 ELISA Assay

Colon tissue was homogenized in cold PBS and centrifuged at 15,000 rpm for 10 min to obtain the supernatant for ELISA assay. The cytokine levels of IL-6 and TNF-α were measured by ELISA kit according to the manufacturer's instructions.

### 1.1.7 RT-PCR

The total RNA from colon tissue was extracted by TRIzol reagent (Thermo Fisher Scientific, USA) according to the user guide. The extracted RNA was then reverse transcribed into cDNA by PrimeScript RT Master Mix (Takara Bio Inc., USA). After that, TB Green Premix Ex Taq (Takara Bio Inc., USA) was used for real-time PCR (QuantStudio™ 7 Flex Real-Time PCR System, Thermo Fisher Scientific). The relative mRNA expression was determined by the threshold cycle (−2ΔΔCt) method, normalizing to β-actin as an endogenous control. The primer sequences used in this study are provided in Table 2.

TABLE 2
Primer information

| | | SEQ | | SEQ |
|---|---|---|---|---|
| Gene | | ID | | ID |
| name | Forward primer | No: | Reverse primer | No: |
| zo-1 | GCCGCTAAGAGCAC | 1 | GCCCTCCTTTTAACA | 2 |
| | AGCAA | | CATCAGA | |
| occludin | TTGAAAGTCCACCT | 3 | CCGGATAAAAAGAGT | 4 |
| | CCTTACAGA | | ACGCTGG | |
| muc2 | AGGGCTCGGAACTC | 5 | CCAGGGAATCGGTAG | 6 |
| | CAGAAA | | ACATCG | |
| β-actin | GGCTGTATTCCCCT | 7 | CCAGTTGGTAACAAT | 8 |
| | CCATCG | | GCCATG | |

### 1.1.8 Statistical Analysis

Statistical analysis was performed by Graphpad Prism 8.0 software, and the results are presented as mean±standard deviation (SD). Statistical significance was determined by one-way ANOVA with Turkey's multiple comparison test. P<0.05 was considered statistically significant.

### 1.2 Results

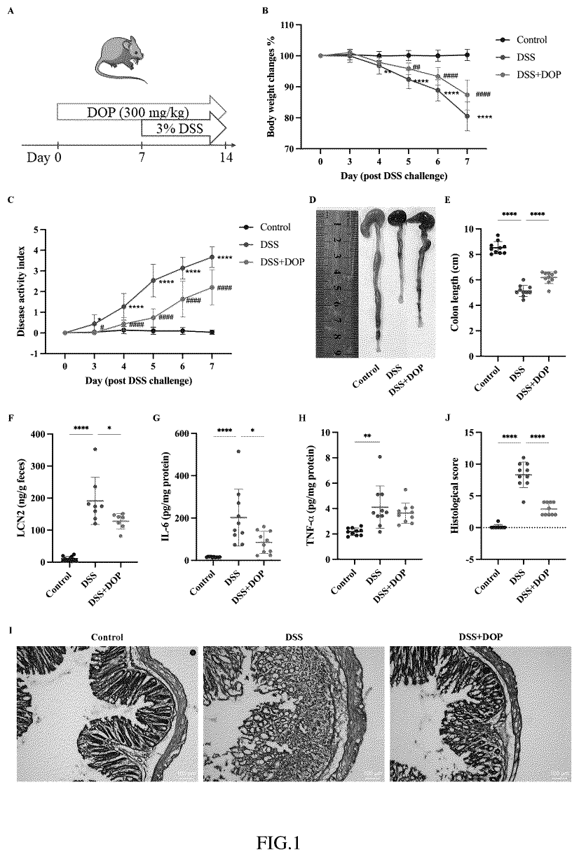

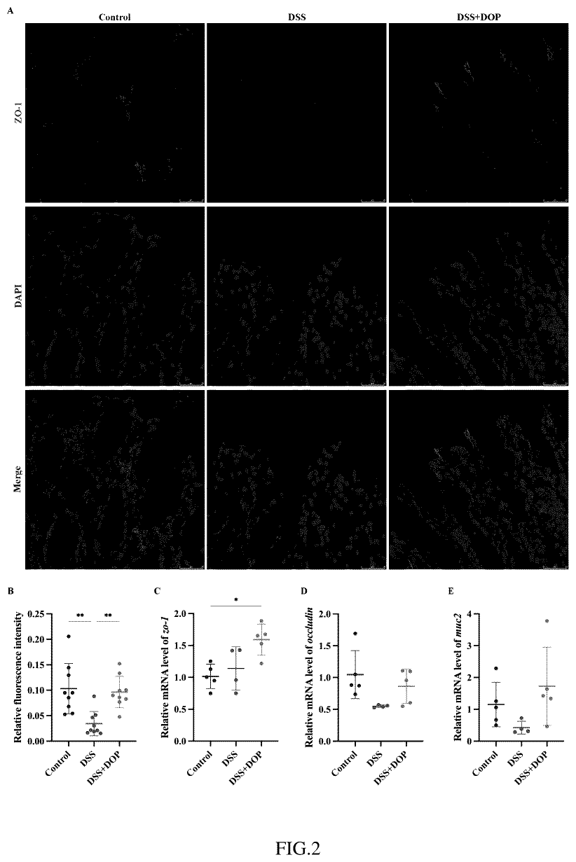

The effect of DOP was evaluated in the DSS-induced colitis mice model (FIG. 1A). Compared to the control group, DSS-treated mice exhibited severe colitis symptoms, including weight loss, stool consistency, and rectal bleeding. In contrast, DOP treatment resulted in a significant improvement in the disease activity index (FIG. 1B-FIG. 1C).

### Colon Length

Colon shortening is one of the parameters to evaluate the severity of DSS-induced colitis. The colon length was reduced in colitis mice when compared to the control group, but increased after DOP intervention. Also, the DSS group displayed bloody intestine, which was improved in the DSS+DOP group (FIG. 2D-FIG. 2E).

### LCN2

LCN2 was suppressed by DOP treatment (FIG. 1F). These findings indicate that DOP alleviated DSS-induced colitis in mice.

### IL-6 and TNF-α

The DSS notably increased IL-6 and TNF-α levels. After DOP intervention, the colonic IL-6 was remarkably reduced, while TNF-α showed a reducing but not significant trend (FIG. 1G-FIG. 1H).

### Colon Histopathological Changes

Colon sections were stained with H&E for the determination of colon histopathological changes.

The colon of the DSS group displayed erosion of epithelium, crypt destruction, and infiltration of inflammatory cells in the submucosa. Notably, DOP treatment ameliorated these conditions with less inflammatory cell infiltration, partially repaired crypt structure, and restoration of the shape of the epithelium (FIG. 1I-J). The results further confirm the protective effect of DOP against DSS-induced colonic injury.

### Tight Junction

Zonula occludens-1 (ZO-1), also known as tight junction protein-1, was reduced in the DSS group, but restored by DOP intervention (FIG. 2A-B). DOP also increased the mRNA expressions of zo-1 and occludin compared to colitis mice although the increase did not reach statistical significance (FIG. 2C-D).

Mucin2 (muc2) mucin is the major component of the mucus layer which lies between the epithelium and lumen. It is produced by the intestinal goblet cells and protects the intestinal epithelium. The data showed that the mRNA expression of muc2 decreased in colitis mice but was restored by DOP treatment (FIG. 2E).

These results indicate that DOP treatment enhanced intestinal barrier function in colitis mice.

### Example 2 DOP Regulates Gut Microbiota Composition and SCFA Generation in Colitis Mice

The dysbiosis of the gut microbial ecosystem is believed to be associated with the occurrence and development of ulcerative colitis. In addition, the production of SCFA has also been reported to be associated with the severity of colitis.

16s rDNA sequencing was used to evaluate the impact of DOP on the intestinal bacterial ecosystem.

In Example 2, the applicant investigated the effect of DOP on the gut microbial community and its related metabolite SCFAs.

SCFAs are the main end-product of gut microbiota during the fermentation of dietary fiber. Acetate, butyrate, and propionate are the most common SCFAs. SCFAs are also reported to be closely related to the health of gut.

### 2.1 Materials and Methods

### 2.1.1 Animal Experiments

To study the interaction between gut microbiota and DOP, different antibiotics were administered to mice: an antibiotic cocktail (ABX) containing 0.5 g/L ampicillin, 0.5 g/L neomycin, 0.5 g/L metronidazole, and 0.25 g/L vancomycin in the drinking water; 0.5 g/L vancomycin in the drinking water; 0.5 g/L neomycin in the drinking water.

In brief, mice were divided into 5 groups: Control group, DOP group, ABX+DOP group, Vancomycin+DOP group, and Neomycin+DOP. All mice (except the Control group) were administered with DOP (200 mg/kg) by oral gavage once daily. Mice in the ABX+DOP group, Vancomycin+DOP group, and Neomycin+DOP group were treated with ABX, vancomycin, and neomycin in drinking water as described above, respectively.

DOP and antibiotic treatment were administered simultaneously for 3 weeks. After mice were sacrificed, the cecal content was collected for further determination.

### 2.1.2 Preparation of FITC-Labeled DOP (F-DOP)

DOP was labeled with FITC. In brief, DOP was dissolved in DMSO with two drops of pyridine. Then, FITC solution and dibutyltin dilaurate were added. The mixture was heated at 98° C. for 2 h, followed by precipitation in ethanol (90% v/v). The precipitate was collected and washed with ethanol (90%, v/v) to remove free FITC dye. The sediment was redissolved in distilled water, dialyzed, and lyophilized to gain F-DOP.

2.1.3 16s rDNA Sequencing

Cecal content was collected, and the 16s rDNA sequencing was conducted by BGI Genomics Co., Ltd. In brief, microbial DNA was extracted and the V3-V4 hypervariable regions of bacteria 16S rRNA gene were amplified with primers for PCR. All PCR products were purified and labeled to finish library construction.

Library size and concentration were detected by Agilent 2100 Bioanalyzer. Qualified libraries were sequenced on the HiSeq platform.

### 2.1.4 SCFA Determination

Cecal content was homogenized in cold PBS and centrifuged at 15,000 rpm for 10 min. The supernatant was collected for SCFA analysis. The content of SCFA was determined.

In brief, the sample was mixed with 2-EBA as the internal standard (IS, 5 μg/mL in methanol). After centrifugation, the supernatant was collected and then mixed with 3-NPH-HCl solution (50 mM, in methanol), EDC.HCl solution (50 mM, in methanol), and pyridine (7% in methanol) at equal volume. After incubating at 37° C. for 30 min, formic acid (0.5% v/v) was added to the mixture and then analyzed by LC-QQQ-MS (Supplementary Method 1).

A mixture of HEPA, HEXA, 2-MVA, 3-MPA, 4-MVA, 2-EBA, VA, 2-MBA, IBA, BA, PA, and AA was used as the SCFA standard. The concentration of the SCFAs was calculated by the area ratios.

2.1.5 High-Performance Gel Permeation Chromatography Coupled with Fluorescence Detector (HPGPC-FLD) Analysis

Cecal content collected from the cecum loop experiment was homogenized in cold PBS and centrifuged at 15,000 rpm for 10 min to obtain supernatant. The supernatant was then heated at 98° C. for 5 min and centrifuged again at 15,000 rpm for 10 min. The supernatant was collected for HPGPC-FLD analysis. Agilent-1100 HPLC system equipped with a fluorescence detector and a TSK GMPWXL column was used for analysis, employing 20 mM ammonium acetate as the mobile phase with the column maintained at 40° C. The FLD was configured to operate with an excitation wavelength of 495 nm and an emission wavelength of 515 nm.

### 2.2 Results

It is demonstrated that DOP promoted the balance of the microbial community and increased SCFAs production in colitis mice.

### Alpha Diversity

Regarding the intestinal bacterial ecosystem, the alpha diversity showed no significant difference between the DSS group and the DSS+DOP group (FIG. 9A-FIG. 9E).

### Principal Component Analysis, PCA

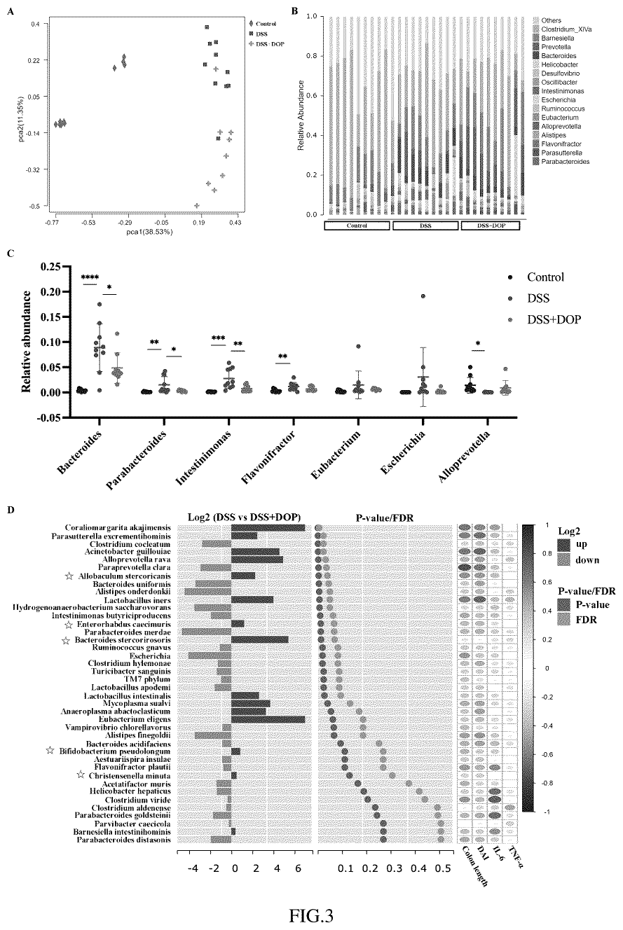

To explore the similarity of the gut microbial structure in the Control group, DSS group, and DSS+DOP group, principal component analysis (PCA) was conducted. The PCA plot revealed a distinct clustering among these 3 groups, indicating differences in the overall microbial composition and structure (FIG. 3A). At the phylum level, Firmicutes and Bacteroidetes were two dominant phyla, representing over 90% of the intestinal bacteria, followed by Proteobacteria. DOP treatment resulted in a higher abundance of Bacteroidetes (FIG. 9F).

At the genus level, the composition of gut microbiota changed after DSS treatment and DOP intervention. To be specific, the relative abundance of Bacteroides, Parabacteroides, Intestinimonas, Flavonifractor, Eubacterium, and Escherichia were increased in the DSS group but reduced by DOP intervention. In contrast, the relative abundance of Alloprevotella was decreased by DSS treatment, then restored in the DSS+DOP group (FIG. 3C).

These data indicate that DOP modulated the gut microbial structure in colitis mice.

### Bacterial Species

The bacterial species between the DSS group and the DSS+DOP group were compared, and it was found that there were 40 species differently abundant in these two groups, with 15 species upregulated and 25 species downregulated by DOP treatment. Correlation analysis of these changed species with colitis-related indexes was performed.

The result showed that Allobaculum stercoricanis, Enterohabdus caecimuris, Bacteroides stercorirosoris, Bifidobacterium pseudolongum, and Christensenella minuta were increased after DOP administration, and that these increases were positively correlated with colon length, but negatively correlated with DAI, IL-6, and TNF-α (FIG. 3D). This indicates that these species may contribute to the anti-colitis effect of DOP.

### SCFAS

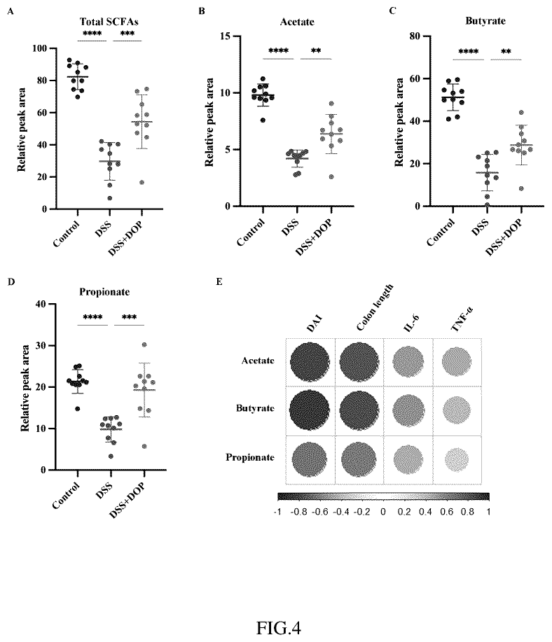

The concentration of SCFAs, including acetate, butyrate, and propionate, were decreased in colitis mice, while DOP treatment increased their levels (FIG. 4A-FIG. 4D). Further correlation analysis showed a negative correlation between SCFAs and DAI, IL-6 expression, and TNF-α expression.

In contrast, the concentration of SCFAs was positively correlated with colon length (FIG. 4E). This means SCFA content may contribute to DOP's anti-colitis effect.

### Example 3 Degradation of DOP Relies on Vancomycin-Sensitive Bacteria

The disrupted gut microbial ecosystem is considered to play a significant role in the development and progression of ulcerative colitis. Differences in gut microbial composition between ulcerative colitis patients and healthy individuals have been observed, with less richness and diversity and a shift towards higher levels of “bad” bacteria in colitis patients.

Gut microbiota are the microorganisms that live in the digestive tract, they are important in the maintenance of the host's health, by affecting the metabolism and regulating the immune function. Evidence has shown that DOP has a regulatory effect on gut microbiota in diverse animal models. The contribution of gut microbiota to DOP's biological activities has not been verified.

DOP is neither absorbed nor degraded in the digestive tract until it reaches the large intestine and is broken down by gut microbiota. Intestinal bacteria may play a key role in the utilization of DOP.

To investigate the interaction between DOP and gut microbiota, different antibiotics were used to figure out what kind of bacteria affected the bioactivity of DOP.

### 3.1 Materials and Methods

### 3.1.1 Animal Experiments

To investigate the role of gut microbiota in the anti-colitis effect of DOP, mice were divided into 5 groups: the Control group, DSS group, DSS+DOP group. Van+DSS group, and Van+DSS+DOP group. Mice in Van+DSS group and Van+DSS+DOP, were treated with vancomycin (0.5 g/L in drinking water) for 26 days. Mice in the DSS+DOP group and Van+DSS+DOP group were administered with DOP (300 mg/kg) by oral gavage once a day starting from day 11 and continued until sacrificed.

For the last week before sacrifice, all mice (except the Control group) were given 3% DSS in drinking water. Body weight, stool consistency, and rectal bleeding were recorded during the DSS challenge. After mice were sacrificed, cecal content and colon were collected.

### 3.1.2 Cecum Loop Experiment

To investigate the degradation of DOP, a cecum loop experiment was conducted. Mice were divided into 3 groups and were treated with normal drinking water (as control mice, Control), 0.5 g/L vancomycin in drinking water (as vancomycin-treated mice, Vancomycin), or 0.5 g/L neomycin in drinking water (as neomycin-treated mice, Neomycin) for 1 week, respectively. Then, mice were anesthetized, followed by an injection of F-DOP (3 mg in 0.3 mL PBS) into the ligated cecum. After that, the cecum was incubated at 37° C. for 1 h. Cecal content was collected for analysis.

### 3.2 Results

Different antibiotics were used to figure out what kind of bacteria affected the bioactivity of DOP.

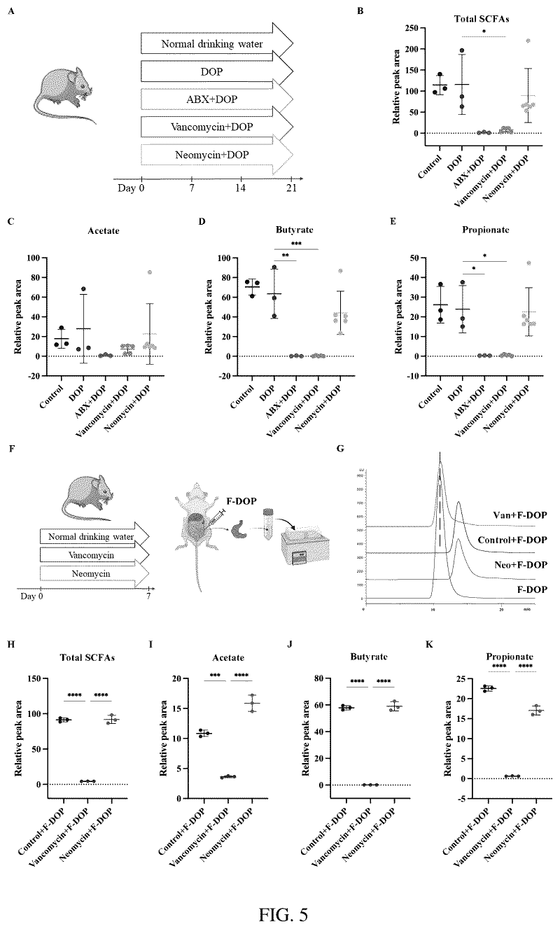

Specifically, first, ABX, vancomycin, and neomycin were used, to deplete all intestinal bacteria, gram-positive bacteria, and gram-negative bacteria, respectively (FIG. 5A).

### SCFAs

As shown in FIG. 5B-FIG. 5E, the production of SCFAs, including acetate, butyrate, and propionate, was significantly inhibited in both ABX-treated mice and vancomycin-treated mice. However, the content of SCFAs detected in neomycin-treated mice was similar to that in the control mice. This finding suggests that the depletion of vancomycin-sensitive bacteria suppressed the production of SCFAs in mice.

### Cecum Loop Experiment

The cecum loop experiment was applied to further verify the correlation between bacteria and the degradation of DOP. F-DOP was used in this experiment to show the integrity of DOP (FIG. 5F). After incubating in the mice's cecum, the retention time of F-DOP remained unchanged in vancomycin-treated mice, indicating that F-DOP was not degraded.

In contrast, the molecular size of F-DOP became smaller when incubated in the cecum of control mice and neomycin-treated mice, which means F-DOP was quickly broken down by the gut microbiota (FIG. 5G).

In line with this result, the generation of SCFAs, including acetate, butyrate, and propionate, was significantly suppressed in vancomycin-treated mice but not in neomycin-treated mice (FIG. 5H-FIG. 5K).

That means vancomycin-sensitive bacteria, but not neomycin-sensitive bacteria are required for the degradation of DOP.

In summery, the results show that antibiotic treatment affects the fermentation of DOP in mice. In particular, vancomycin-sensitive bacteria are responsible for the metabolism of DOP to generate SCFAs. Vancomycin-sensitive bacteria but not neomycin-sensitive bacteria are required for DOP metabolism.

The results suggest that it may be possible to increase the ability of DOP, i.e., to increase SCFAs, by supplementing with Vancomycin-sensitive bacteria, to achieve the treatment of ulcerative colitis.

### Example 4 DOP Fails to Exert Anti-Colitis Effects after Vancomycin Intervention

To further verify the role of vancomycin-sensitive bacteria in the bioactivity of DOP, vancomycin was applied to deplete its target bacteria before the establishment of the colitis model and DOP treatment.

The body weight, the Disease activity index, the colon length, the pro-inflammatory cytokines and the concentration of SCFAs were measured in this Example.

### 4.1 Materials and Methods

### 4.2 Results

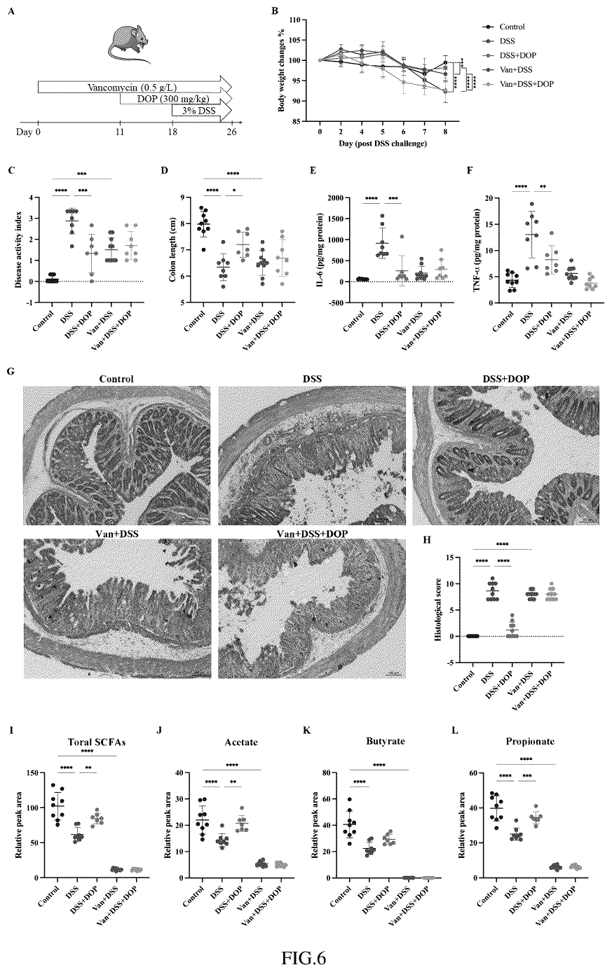

Vancomycin was applied to deplete its target bacteria before the establishment of the colitis model and DOP treatment (FIG. 6A).

### Body Weight and Disease Activity Index

As expected, mice in the DSS group experienced a significant decrease in body weight during the DSS challenge, which was improved by the DOP supplement. In line with this, the DSS-induced elevated disease activity index was reduced by DOP in colitis mice.

Interestingly, vancomycin pretreatment also resulted in weight loss in mice but was milder than the DSS group, which was not improved after DOP treatment.

Vancomycin-treated mice developed colitis-like symptoms following DSS treatment. However, the disease activity index showed no difference between the Van+DSS group and the Van+DSS+DOP group (FIG. 6B-FIG. 6C).

### Colon Length

The colon length in the DSS+DOP group was increased when compared to the DSS group. Colon shortening was also observed in the Van+DSS group, while DOP intervention did not improve colon length (FIG. 6D).

### Pro-Inflammatory Cytokines

The expression of pro-inflammatory cytokines IL-6 and TNF-α was increased upon DSS challenge but was reversed by DOP treatment. In contrast, DOP treatment did not change the expression of IL-6 and TNF-α after vancomycin pretreatment (FIG. 6E-F).

These findings indicate that vancomycin pretreatment abolished the anti-colitis effects of DOP, thereby further confirming the importance of vancomycin-sensitive bacteria in the anti-colitis effect of DOP.

### Histopathological Changes

histopathological changes were observed in colitis mice, with damaged crypts and disruption of epithelial lining. The loss of crypts and destroyed epithelium were also seen in the Van+DSS group, however, crypts of normal shape and a well-organized epithelial layer were observed in the group treated with DOP. In contrast, vancomycin-treated colitis mice did not exhibit these improvements after DOP treatment, suggesting that the beneficial effect of DOP is mediated by vancomycin-sensitive bacteria (FIG. 6G-FIG. 6H).

### SCFAs

The concentration of SCFAs were measured in these groups. As shown in FIG. 6I-FIG. 6L, the content of SCFAs (acetate, butyrate, and propionate) was restored by DOP treatment in colitis mice. The vancomycin treatment led to a minimal level of SCFAs in the cecum of colitis mice, which was not increased by DOP. This result further demonstrates that DOP could boost the generation of SCFA in colitis mice, and this boosting was suppressed by vancomycin treatment.

These findings suggest that dietary fibers may become useless when administrated together with antibiotics, but this conflict is avoidable if people select appropriate antibiotics.

Therefore, it is recommended that probiotics be used in combination with dietary fiber. Specifically, it is recommended that DOP be used in combination with vancomycin-sensitive bacteria associated with its metabolism.

### Example 5 Vancomycin-Sensitive Bacteria Encode Mannan Endo-1,4-β-Mannosidase to Degrade DOP

As a macromolecular polymer, DOP is not degradable by the host due to the lack of related enzymes within the mammal genome.

The applicant hypothesized that upon exposure to DOP, the gut microbiota undergoes adaptive changes, resulting in the enrichment of specific bacterial populations capable of producing enzymes that degrade DOP. If so, after being treated with DOP, the bacteria within the DSS+DOP group would show stronger activity in degrading DOP.

The expression and enzyme activity of EC 3.2.1.78 in control mice and vancomycin-treated mice were verified.

### 5.1 Materials and Methods

### 5.1.1 Oligosaccharide Analysis

The p-aminobenzoic ethyl ester (ABEE) derivatization method was used for oligosaccharide analysis. The sample was incubated with ABEE, sodium cyanoborohydride (NaBH₃CN), and glacial acetic acid for 2 h at 65° C. The reaction was quenched by adding Milli Q and cooled at 4° C. for 10 min. Subsequently, the mixture was extracted 5 times with diethyl ether. The water layer was collected and dried using nitrogen gas, then dissolved in 60% methanol for LC-qTOF-MS analysis.

### 5.1.2 Bacteria Culture and Protein Extraction

Bifidobacterium pseudolongum (DSM20092) was obtained from DSMZ-German collection of microorganisms and cell cultures GmbH. The B. pseudolongum was cultured in the MRS medium (Table 3) using a shaking incubator at 37° C. The culture medium was placed in an anaerobic chamber (Bactron) (N₂—CO₂-H₂, 85%-10%-5%) to remove oxygen. All the procedures were conducted under the anaerobic condition.

TABLE 3
Histological score

| | 0 | 1 | 2 | 3 |
|---|---|---|---|---|
| Inflammatory | Inflammatory | Increase of | Inflammatory | Inflammatory |
| cells | cells scattered | inflammatory | cells aggregate | cell transmural |
| infiltration | in lamina | cells in lamina | and invade the | |
| | propria | propria | submucosa | |
| | mucosa | mucosa | | |
| Mucosal | The mucosa is | Dispersed | Surface | Mucosal |
| injury | regular and | epithelial | mucosal | damage |
| | normal in | injury | erosion or | extends to the |
| | shape. | | focal ulcer | deep intestinal |
| | | | | wall |
| Glandular | The shape of | ⅓ area injury | ⅔ area injury | Only |
| fossa injury | glandular | in base | in base | superficial |
| | fossa is | | | epithelial cells |
| | normal | | | are intact |
| The degree of | Complete | Nearly | Mucosal | Only the |
| regeneration | repair or | complete | epithelium | superficial |
| and repair of | normal tissue | regenerative | regenerate, but | epithelial cells |
| mucosal | | repair | crypt is absent | are not |
| epithelium | | | | damaged |

The cultured bacteria were centrifuged at 4,000 rpm for 15 min at 4° C. The cell pellets were collected and washed with PBS. After that, the bacterial cell was lysed by ultrasonication. After centrifugation at 15,000 rpm for 10 min, the supernatant was collected in a new tube as bacterial protein.

### 5.1.3 Zymogram

The cecal content was collected from control mice (Control, C) and vancomycin-treated mice (Vancomycin, V). Cecal content was homogenized in cold PBS, then centrifuged at 4,000 rpm, the supernatant was collected. The protein level was measured using BCA assay kit. A total protein of 10 μg was separated by SDS-PAGE gels (containing 2.5 mg DOP/gel). After that, the gel was incubated in 2.5% Triton X-100 at room temperature for 1 h to remove SDS, and then immersed in 50 mM citrate buffer (pH 5.5) at room temperature for 1 h. The gel was stained in 0.5% Congo Red for 30 min and washed with 1M NaCl until the bands developed.

### 5.2 Results

### Capability to Break Down F-DOP

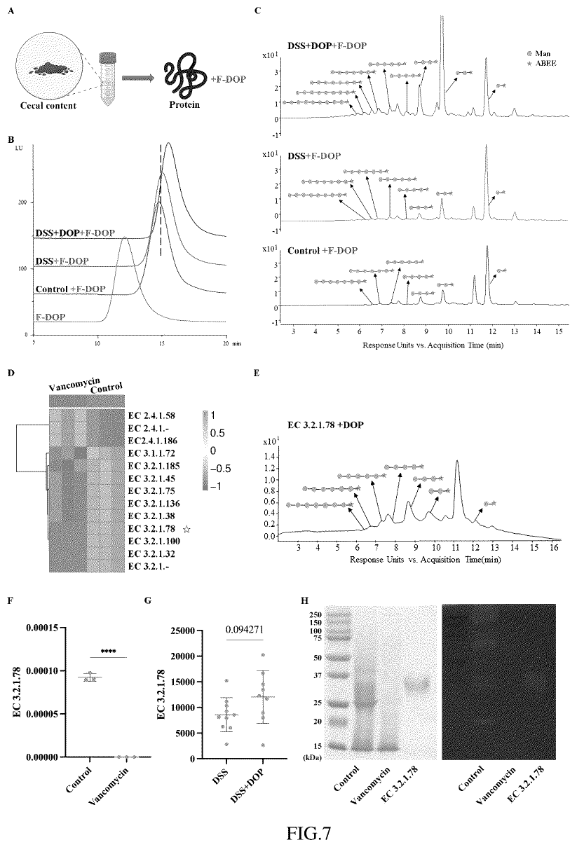

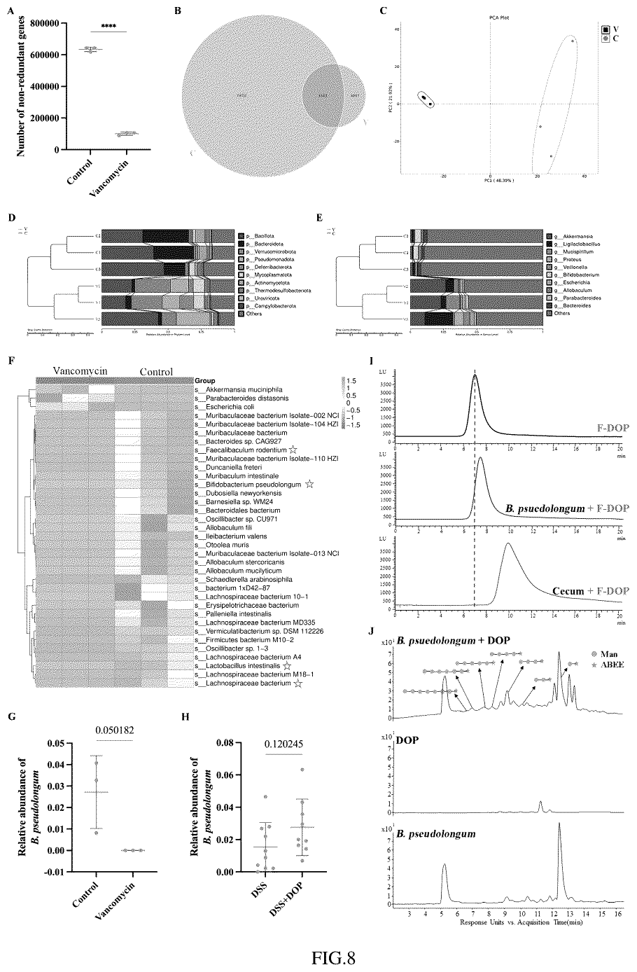

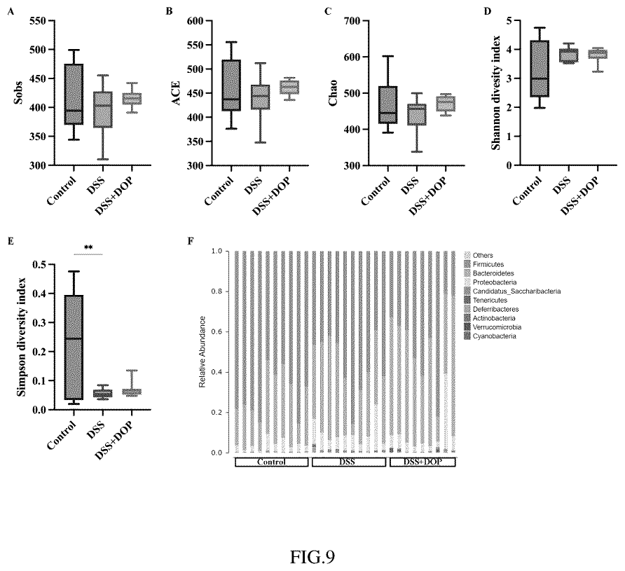

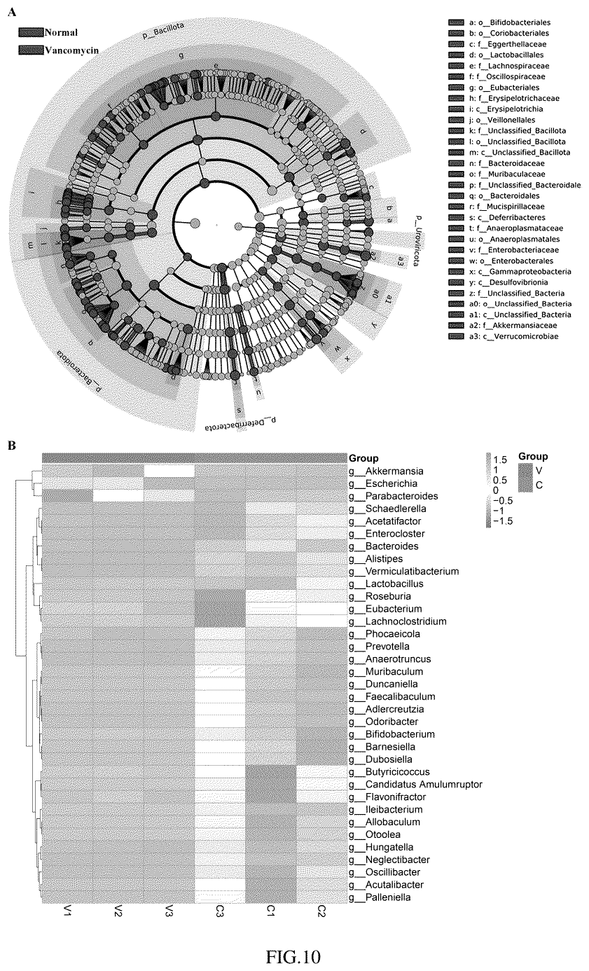

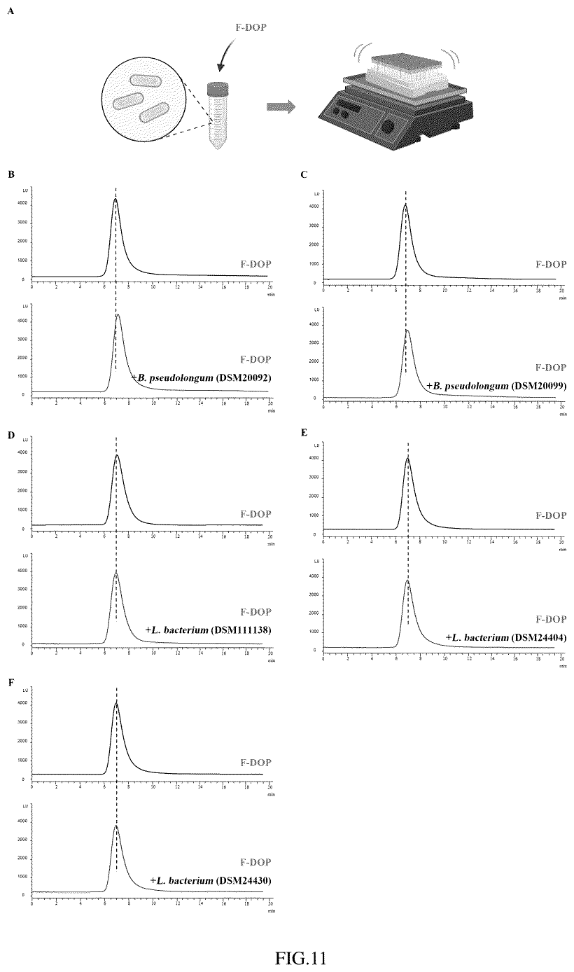

To test the applicant's hypothesis, the bacterial protein was extracted from the Control group, DSS group, and DSS+DOP group and then incubated with F-DOP in order to determine its capability to break down F-DOP (FIG. 7A).

The results showed that F-DOP was degraded in all 3 groups within 6 h, however, at different rates. Specifically, the DSS+DOP+F-DOP group exhibited a longer retention time compared to the control group and DSS+F-DOP group (FIG. 7B). This result suggested that the bacterial protein extracted from the DOP-treated gut microbiota (DSS+DOP group) had a remarkable ability to degrade DOP.

### Oligosaccharide Analysis

The oligosaccharide analysis showed that F-DOP was degraded into mannan oligosaccharides. Specifically, the DSS+DOP+F-DOP group generated more mannan oligosaccharides (increased peak area of the annotated oligosaccharides), including mannan disaccharide, mannan trisaccharide, mannan tetrasaccharide, mannan pentasaccharide, mannan hexasaccharide, mannan heptasaccharide, mannan octasaccharide, mannan nonasaccharide (mannan oligosaccharides that contain 2 to 9 mannose units) (FIG. 7C).

These results suggest a synergistic interplay between DOP and the intestinal bacteria, in which the presence of DOP induces the gut microbiota to produce specialized enzymes required for its effective breakdown.

The applicant compared the enzyme profiles of the control mice and vancomycin-treated mice and found a series of carbohydrate-active enzymes (CAZymes) differed.

CAZymes are the mediators for gut microbiota to degrade polysaccharides (Wardman et al., 2022). DOP, as a complex carbohydrate, can be degraded by bacteria-derived CAZymes. CAZymes are a diverse group of enzymes that are involved in the metabolism and modification of carbohydrates, these enzymes include glycoside hydrolases (GHs), glycosyltransferases (GTs), polysaccharide lyases (PLs), carbohydrate esterases (CEs), and auxiliary activities (AAs).

Within the CAZymes group, the applicant found that, after vancomycin treatment, the family of GHs (EC 3.2.1.-) was suppressed while the family of GTs (EC 2.4 . . . ) was upregulated (FIG. 7D).

EC 3.2.1.78, commonly referred to as mannan endo-1.4-β-mannosidase or β-mannanase, is an enzyme that capable of randomly hydrolyzing the β-1,4-mannosidic bonds within the mannans. Oligosaccharide analysis revealed that EC 3.2.1.78 catalyzed DOP to yield mannan oligosaccharides (FIG. 7E).

Moreover, data also showed that the expression of EC 3.2.1.78 was significantly decreased in vancomycin-treated mice compared to the control mice (FIG. 7F). Furthermore, its expression was also found relatively higher in the DSS+DOP group compared to the DSS group, indicating a better DOP-hydrolyzing ability in the DSS+DOP group (FIG. 7G). This finding may explain the stronger DOP-degrading ability detected in the bacterial protein of the DSS+DOP group.

### Expression and Enzyme Activity of EC 3.2.1.78

The bacterial protein was extracted from the cecal content of control mice and vancomycin-treated mice, respectively.

Coomassie-stained gel of the bacterial protein from the control mice displayed a series of protein bands, while most of the protein bands were diminished in the gels of vancomycin-treated mice (FIG. 7H, the left panel). Moreover, EC 3.2.1.78 was diminished in vancomycin-treated mice as shown in zymography which examined the enzyme activity of EC 3.2.1.78. The band of EC 3.2.1.78 shown in control mice disappeared in the vancomycin-treated mice, where it appeared as clear zones of lysis against a background of unhydrolyzed DOP (FIG. 7H, the right panel).

These results suggest that the CAZyme EC 3.2.1.78 which degrades DOP is encoded by vancomycin-sensitive bacteria.

### Example 6 Vancomycin-Sensitive Bifidobacterium pseudolongum is Involved in the Degradation of DOP

To further identify the specific bacteria that is responsible for DOP metabolism, metagenomic analysis was used to investigate the intestinal microbial structural differences between control mice and vancomycin-treated mice in this Example.

The changed intestinal bacteria following vancomycin treatment were compared and shortlisted 5 bacteria that may contribute to DOP's activity. Further, the applicant focused on 2 candidates (B. pseudolongum and L. bacterium) that potentially produce EC 3.2.1.78, and determined their ability to degrade DOP.

### 6.1 Materials and Methods

B. pseudolongum and L. bacterium were cultured under the anaerobic condition, with F-DOP supplemented in the medium. F-DOP in the blank culture medium was used as a control. The incubation was conducted in a shaking incubator.

### 6.1.1 Shotgun Metagenomic Sequencing

Cecal content was collected from control mice (as Control group) and vancomycin-treated mice (as Vancomycin group), respectively.

The shotgun metagenomic sequencing was conducted by Novegene Bioinformatics Technology Co., Ltd. DNA was extracted from the cecal content for library construction. The library size and concentration were detected by Agilent 2100 Bioanalyzer. The library was sequenced on the Illumina PE500 platform.

### 6.2 Results

### Intestinal Microbial Structural Differences

Metagenomic analysis was used to investigate the intestinal microbial structural differences between control mice and vancomycin-treated mice. As shown in FIG. 8A, the number of non-redundant genes was remarkably reduced after vancomycin treatment.

The Venn diagram analysis revealed that a total of 797,135 OTU reads were detected in control mice, while only 122,330 OTU reads were detected in vancomycin-treated mice, with a substantial overlap of 81,413 OTUs shared between these 2 groups (FIG. 8B).

### PCA

PCA analysis shows distinct clustering patterns indicative of differences in microbial composition between control mice and vancomycin-treated mice. Especially, the control mice, with relatively more OTUs, had a more complicated and diverse microbial profile, which made the PCA plot more widely distributed and variable. In contrast, the PCA plot of vancomycin-treated mice was more concentrated and had closer clustering (FIG. 8C).

The clustering tree represents the relationships and similarities between samples based on their microbial composition. The clustering results reveal that microbial community structures are more similar within groups, with samples within each cluster sharing a common phylum-level microbial profile. The phylum levels of Firmicutes and Bacteroidetes were decreased, while the relative abundance of Verrucomicrobiota and Proteobacteria were increased in vancomycin-treated mice compared to the control mice (FIG. 8D).

At the genus level, the relative abundance of Akkermansia, Ligilactobacillus, Mucispirillum, Proteus, Veillonella, Escherchia and Parabacteroides were increased after vancomycin treatment. In contrast, the relative abundance of Bifidobacterium, Allobaculum, and Bacteroides were reduced by vancomycin (FIG. 8E). Lefse analysis and Metastat analysis was applied to reveal significant differences in bacterial taxa abundance between these two groups. The result shows that the control mice had a higher number of enriched bacteria compared to vancomycin-treated mice (FIG. 10A-FIG. 10B).

Then, the Metastat analysis was applied to compare the most significantly different species between the control mice and vancomycin-treated mice. The applicant found a total of 35 species were significantly changed within these two groups, with 3 species upregulated and 32 species downregulated in the vancomycin group (FIG. 8G).

By searching on the National Center for Biotechnology Information, the bacterium Bifidobacterium pseudolongum (GenBank: RYQ50456.1) and Lachnospiraceae bacterium (GenBank: GKH55604.1) were found to have the potential to express mannan endo-1,4-β-mannosidase.

B. pseudolongum is a gram-positive and nonmotile bacterium belonging to the Actinobacteria phylum, and it is commonly found in the gut microbiota of mammals.

### Ability to Degrade F-DOP

The applicant obtained different strains (DSM20092 and DSM20099 for B. pseudolongum, DSM111138, DSM 24404, and DSM 24430 for Lachnospiraceae bacterium) of these two bacteria and cultured them in the presence of F-DOP to determine their ability to degrade F-DOP (FIG. 11A).

The result showed that the fluorescence signal of F-DOP remained unchanged after incubation with L. bacterium, but shifted after incubation with B. pseudolongum (FIG. 11B-FIG. 11F). This indicates that B. pseudolongum is capable of breaking down F-DOP. The relative abundance of B. pseudolongum was decreased in vancomycin-treated mice compared to the control mice (FIG. 8G).

Data showed that the relative abundance of B. pseudolongum was relatively higher following DOP treatment in colitis mice (FIG. 8H), indicating its role in DOP metabolism. The presence of this bacterium was associated with the anti-colitis effect of DOP (FIG. 3D).

Given this evidence, B. pseudolongum is the bacterial agent responsible for the degradation and biological activity of DOP.

### Example 7 B. pseudolongum Contributes to the Beneficial Effects of DOP

While the abundance of B. pseudolongum can vary among individuals, DOP supplementation could potentially promote its growth to increase its abundance or the growth of other functionally similar mannan-degrading bacteria.

Several studies have confirmed the presence of B. pseudolongum in human gut microbiota. This indicates that the bacterium, and by extension the mechanisms we've elucidated, potentially hold relevance for human health.

To further investigate the effect of B. pseudolongum on DOP, the bacterial protein was extracted from B. pseudolongum and cecal content from the control mice, respectively. The extracted proteins were used to examine their catalyzing ability on DOP degradation. 7.1 Results Ability to degrade F-DOP

As shown in FIG. 8I, the incubation of bacterial protein from B. psueolongum with F-DOP also resulted in the changed fluorescent signaling compared to the F-DOP, indicating its degradation. Interestingly, the fluorescent signal of F-DOP exhibited a more significant change after incubation with bacterial protein from the cecal content compared to the B. pseudolongum+F-DOP. The cecal content had a better ability to metabolize DOP than the species B. pseudolongum alone.

### Oligosaccharide Analysis

Oligosaccharide analysis also revealed that incubation of B. pseudolongum protein with DOP resulted in an increased amount of mannan oligosaccharides, including mannan disaccharide, mannan trisaccharide, mannan tetrasaccharide, mannan pentasaccharide, mannan hexasaccharide, and mannan heptasaccharide (containing 2 to 7 mannose units) (FIG. 8J) (FIG. 12A-F).

That means B. pseudolongum can break down DOP to yield mannan oligosaccharides, which can be further fermented into SCFAs. Collectively, these findings demonstrate that B. pseudolongum has the catalyzing ability to degrade DOP into mannan oligosaccharides, which contributes to the beneficial effect of DOP.

In particular, the vancomycin-sensitive bacterium B. pseudolongum, is capable of degrading DOP into mannan oligosaccharides and contributes to the beneficial effects of DOP.

Different strains of the same bacterial species exhibited varying effects on DOP. B. pseudolongum, Isolating bacterial strains from gut microbiota may help preserve the reactivity of the bacteria of interest. Data shows B. pseudolongum DSM20092 and B. pseudolongum DSM20099 could be used to prepare compositions to enhance the beneficial effects of DOP.

The cultured bacterium in the experiments has two limitations: (i) insufficient expression or efficacy of CAZymes: (ii) contains carbohydrates, digested proteins, and sodium in medium. Therefore, the applicant extracted the bacterial protein from the cultured bacteria. This can help to enrich the enzymes and eliminate the impact of the culture medium.

Combined with its association with DOP's anti-colitis effects, the applicant consider the vancomycin-sensitive bacterium B. pseudolongum is the mediator for DOP's bioactivities.

The results demonstrate the significant role of vancomycin-sensitive bacteria in the metabolism and biological activities of DOP. These bacteria produce the enzyme EC 3.2.1.78, responsible for the breakdown of DOP. The activity of this enzyme can be enhanced by DOP intervention, leading to improved DOP metabolism.

Accordingly, EC 3.2.1.78 may also be used to prepare compositions that enhance the beneficial effects of DOP.

### Example 8 Compositions Containing DOP and B. pseudolongum

### 8.1 Preparation of the Composition

By mixing the lyophilized powder of DOP prepared in Example 1 with the cell of B. pseudolongum, a composition containing DOP and B. pseudolongum was obtained.

### Example 9 Compositions Containing DOP and B. pseudolongum Extract

### 9.1 Preparation of the Composition

By mixing the lyophilized powder of DOP prepared in Example 1 with powder of B. pseudolongum extract prepared in Example 5, a composition containing DOP and B. pseudolongum extract was obtained.

### Example 10 Compositions Containing DOP and EC 3.2.1.78

### 10.1 Preparation of the Composition

By mixing the lyophilized powder of DOP prepared in Example 1 with powder of EC 3.2.1.78, a composition containing DOP and EC 3.2.1.78 was obtained.

### REFERENCE

1. Le Berre, C., Honap, S., & Peyrin-Biroulet, L. (2023). Ulcerative colitis. Lancet (London, England), 402 (10401), 571-584. https://doi.org/10.1016/S0140-6736 (23) 00966-2
2. Wardman, J. F., Bains, R. K., Rahfeld, P., & Withers, S. G. (2022). Carbohydrate-active enzymes (CAZymes) in the gut microbiome. Nature Reviews Microbiology, 20 (9), 542-556. https://doi.org/10.1038/s41579-022-00712-1
3. Wong, T. L., Li, L. F., Zhang, J. X., Zhang, Q. W., Zhang, X. T., Zhou, L. S., Fung, H. Y., Feng, L., Cheng, H. Y., Huo, C. Y., Liu, M., Bao, W. R., Wu, W. J., Lai, C. H., Bai, S. P., Nie, S. P., Puno, P. T., Lau, C. B. S., Leung, P. C., . . . . Sun, H. D. (2023). Oligosaccharide analysis of the backbone structure of the characteristic polysaccharide of Dendrobium officinale. Food Hydrocolloids, 134, 108038. https://doi.org/10.1016/j.foodhyd.2022.108038
4. He, T. B., Huang, Y. P., Yang, L., Liu, T. T., Gong, W. Y., Wang, X. J., et al. (2016). Structural characterization and immunomodulating activity of polysaccharide from Dendrobium officinale. International Journal of Biological Macromolecules, 83, 34-41.
5. Hua, Y., Zhang, M., Fu, C., & Chen, Z. (2004). Structural characterization of a 2-O-acetylglucomannan from Dendrobium officinale stem. Carbohydrate Research, 339, 2219-2224.
6. Kuang, M. T., Li, J. Y., Yang, X. B., Yang, L., Xu, J. Y., Yan, S., et al. (2020). Structural characterization and hypoglycemic effect via stimulating glucagon-like peptide-1 secretion of two polysaccharides from Dendrobium officinale. Carbohydrate Polymers, 241, Article 116326.
7. Luo, Q., Tang, Z., Zhang, X., Zhong, Y., Yao, S., Wang, L., et al. (2016). Chemical properties and antioxidant activity of a water-soluble polysaccharide from Dendrobium officinale. International Journal of Biological Macromolecules, 89, 219-227.
8. Wei, W., Feng, L., Bao, W. R., Ma, D. L., Leung, C. H., Nie, S. P., et al. (2016). Structure characterization and immunomodulating effects of polysaccharides isolated from Dendrobium officinale. Journal of Agricultural and Food Chemistry, 64 (4), 881-889.
9. Xing, X., Cui, S. W., Nie, S., Phillips, G. O., Goff, H. D., & Wang, Q. (2014). Study on Dendrobium officinale O-acetyl-glucomannan (Dendronan®): Part I. Extraction, purification, and partial structural characterization. Bioactive Carbohydrates and Dietary Fibre, 4 (1), 74-83.
10. Yang, J., Chen, H., Nie, Q., Huang, X., & Nie, S. (2020). Dendrobium officinale polysaccharide ameliorates the liver metabolism disorders of type II diabetic rats. International Journal of Biological Macromolecules, 164, 1939-1948.

1. A use of vancomycin-sensitive bacteria, comprising making therapeutically effective doses of vancomycin-sensitive bacteria available in the intestines of subjects in need thereof, for increasing intestinal absorption of Dendrobium polysaccharide (DP) in subjects in need thereof.

2. The use according to claim 1, wherein
the vancomycin-sensitive bacteria includes one or more of the following genera: Schaedlerella, Acetatifactor, Enterocloster, Bacteroides, Alistipes, Vermiculatibacterium, Lactobacillus, Roseburia, Eubacterium, Lachnoclostridium, Phocaeicola, Prevotella, Anaerotruncus, Muribaculum, Duncaniella, Faecalibaculum, Adlercreutzia, Odoribacter, Bifidobacterium, Barnesiella, Dubosiella, Butyricicoccus, Candidatus, Amulumruptor, Flavonifractor, Ileibacterium, Allobaculum, Otoolea, Hungatella, Neglectibacter, Oscillibacter, Acutalibacter, and Palleniella.

3. The use according to claim 1, wherein the DP is Dendrobium officinale polysaccharide (DOP), the backbone structure of the DOP is β-D-1,4-mannan.

4. The use according to claim 1, wherein the vancomycin-sensitive bacteria express glycoside hydrolase EC 3.2.1.78.

5. The use according to claim 1, wherein making therapeutically effective doses of vancomycin-sensitive bacteria available in the intestines of subjects in need thereof comprises one or more of the following:
discontinuing vancomycin;
administering DOP;
supplementing with probiotics; and
restoring the subject's intestinal flora balance.

6. The use according to claim 1, wherein the subjects in need thereof have an inflammatory bowel disease.

7. A composition for increasing gut health, comprising
(i) Dendrobium polysaccharide (DP); and
(ii) live, inactivated, dead, or lysed bacteria of vancomycin-sensitive bacteria, etc.

8. The composition according to claim 7, wherein increasing gut health comprises one or more of the following:
restoring dysbiosis in a subject's gut microbial ecosystem, increasing a subject's intestinal short-chain fatty acid (SCFA) production, increasing mannan oligosaccharide production, alleviating colitis-like symptoms, restoring colon length, suppressing intestinal inflammation, enhancing intestinal barrier function, and decreasing colonic injury.

9. The composition according to claim 7, wherein
the vancomycin-sensitive bacteria includes one or more of the following genera: Schaedlerella, Acetatifactor, Enterocloster, Bacteroides, Alistipes, Vermiculatibacterium, Lactobacillus, Roseburia, Eubacterium, Lachnoclostridium, Phocaeicola, Prevotella, Anaerotruncus, Muribaculum, Duncaniella, Faecalibaculum, Adlercreutzia, Odoribacter, Bifidobacterium, Barnesiella, Dubosiella, Butyricicoccus, Candidatus, Amulumruptor, Flavonifractor, Ileibacterium, Allobaculum, Otoolea, Hungatella, Neglectibacter, Oscillibacter, Acutalibacter, and Palleniella.

10. The composition according to claim 7, wherein
the vancomycin-sensitive bacteria is Bifidobacterium pseudolongum.

11. The composition according to claim 10, wherein Bifidobacterium pseudolongum is selected from the group of DSM20092 and DSM20099.

12. The composition according to claim 7, wherein the DP is Dendrobium officinale polysaccharide (DOP), the backbone structure of the DOP is β-D-1,4-mannan.

13. A method for treating an inflammatory bowel disease in a subject, comprising:
i) administering a therapeutically effective dose of DP;
ii) prior to step i,
making therapeutically effective doses of vancomycin-sensitive bacteria available in the intestines of the subject, wherein the vancomycin-sensitive bacteria express glycoside hydrolase EC 3.2.1.78; or
administering a therapeutically effective dose of bacterial proteins of the vancomycin-sensitive bacteria, and/or glycoside hydrolase EC 3.2.1.78 to the subject.

14. The method according to claim 13, wherein the making therapeutically effective doses of vancomycin-sensitive bacteria available in the intestines of the subject comprises:
administering a therapeutically effective dose of vancomycin-sensitive bacteria to the subject.

15. The method according to claim 13, wherein the inflammatory bowel disease is selected from:
indeterminate colitis, ulcerative colitis and Crohn's disease.

16. The method according to claim 13, wherein
the vancomycin-sensitive bacteria is Bifidobacterium pseudolongum.

17. The method according to claim 16, wherein Bifidobacterium pseudolongum is selected from the group of DSM20092 and DSM20099.

18. The method according to claim 13, wherein the making therapeutically effective doses of vancomycin-sensitive bacteria available comprises one or more of the following:
discontinuing vancomycin;
administering DOP;
supplementing with probiotics; and
restoring the subject's intestinal flora balance.

19. The method according to claim 13, wherein before step i) and/or step ii), the method further comprises step iii),
iii) the testing step, testing for the presence of a therapeutically effective dose of vancomycin-sensitive bacteria in the intestinal tract of a subject in need thereof.

20. The method according to claim 13, wherein the restoring the subject's intestinal flora balance comprises increasing the level of vancomycin-sensitive bacteria in the intestinal tract.


SEQUENCE LISTING (8 sequences)

SEQ ID NO: 1 - DNA, 19 bp, synthetic construct
  source 1..19: mol_type = other DNA
gccgctaagagcacagcaa

SEQ ID NO: 2 - DNA, 22 bp, synthetic construct
  source 1..22: mol_type = other DNA
gccctccttttaacacatcaga

SEQ ID NO: 3 - DNA, 23 bp, synthetic construct
  source 1..23: mol_type = other DNA
ttgaaagtccacctccttacaga

SEQ ID NO: 4 - DNA, 22 bp, synthetic construct
  source 1..22: mol_type = other DNA
ccggataaaaagagtacgctgg

SEQ ID NO: 5 - DNA, 20 bp, synthetic construct
  source 1..20: mol_type = other DNA
agggctcggaactccagaaa

SEQ ID NO: 6 - DNA, 21 bp, synthetic construct
  source 1..21: mol_type = other DNA
ccagggaatcggtagacatcg

SEQ ID NO: 7 - DNA, 20 bp, synthetic construct
  source 1..20: mol_type = other DNA
ggctgtattcccctccatcg

SEQ ID NO: 8 - DNA, 21 bp, synthetic construct
  source 1..21: mol_type = other DNA
ccagttggtaacaatgccatg